(19) United States
(12) Patent Application Publication
(10) Pub. No.: US 2007/0099234 A1
(43) Pub. Date: May 3, 2007

(54) Specific detection of chitin using chitin-binding domain

(75) Inventors: Yinhua Zhang, North Reading, MA (US); Larry McReynolds, Beverly, MA (US)

Correspondence Address: HARRIET M. STRIMPEL; NEW ENGLAND BIOLABS, INC., 240 COUNTY ROAD, IPSWICH, MA, 01938-2723, US

(73) Assignee: NEW ENGLAND BIOLABS, INC., IPSWICH, MA (US)

(21) Appl. No.: 10/562,183

(22) PCT Filed: Jun. 7, 2004
(86) PCT No.: PCT/US04/17979

§ 371 Date: Dec. 22, 2005

Related U.S. Application Data
(60) Provisional application No. 60/483,279, filed on Jun. 27, 2003.

(51) Int. Cl. G01N 33/53

(52) U.S. Cl. 435/7.1

(57) ABSTRACT

Methods and kits are provided for detecting chitin in biological samples using chitin-binding domain (CBD).

### BACKGROUND

Chitin is one of the most abundant biopolymers in nature. Although chitin content varies significantly among different organisms, it is widely distributed among invertebrates including arthropods, nematodes, crustaceans, fungi and some protozoa. It functions generally as an impermeable layer and mechanical support in the cell wall and exoskeleton. For example, when budding yeast cells divide, the locations on the cell wall where the daughter cells detach leave a scar consist of chitin and detection of such budding scar have been used evidence of cell division and location of cell separation. In some invertebrates such as in nematodes, chitin is known to play a role in the eggshell to protect developing embryos from the hostile environment and provide a rigid housing.

Since chitin is absent in vertebrates and plants, detection of chitin in these organisms provides a method of diagnosis of infection or contamination by chitin-containing organisms. The availability of a specific and sensitive chitin-detecting reagent can also further facilitate investigations into the properties of chitin and its associated structures as well as the location and function of chitin in selected chitin containing organisms.

Fungal infections are a major health problem, particularly in immunocompromised patients such as those with acquired immune deficiency syndrome (AIDS) or patients receiving a bone marrow transplant. Due to their compromised immune systems, these patients are under threat of fungal infection, which is less common in people with normal immune systems. Such infection is often termed opportunistic infection and they are the main direct causes of morbidity and mortality in AIDS patients. In fact, several fungal infections have been used as indicators for AIDS: candidosis (caused by Candida, which is a yeast that contains chitin in its cell wall) of the esophagus, trachea, bronchi, or lungs; and meningeal cryptococcosis (Centers for Disease Control and World Health Organization, 1988) Thus, accurate and timely diagnosis of fungal infection is an important step before proper and timely medical intervention.

There is also an unfilled need for better means to diagnose fungal infections in plant tissues, both in growing plants and in harvested crops and foods. Direct economic losses in agriculturally important crops caused by fungal infections cost billions of dollars annually.

A large number of naturally occurring chitinases have been described that bind to and degrade chitin. Some of these chitinases have been isolated, labeled and used for detection of chitin. (Benjaminson, M. A. Stain Technology 44:27-31 (1969), Chamberland et al., Histochem. J. 17:313-321 (1985), Benhamou, et al. Biology of the Cell 67:341-50 (1989)).

In addition, 2 chitin-inducible proteins have been recently identified, namely, Chitovibrin (Gildemeister et al. Glyconjugate Journal 11:518-526 (1994)) and Chitinase VP1 (Laine, R. A. et al. U.S. Pat. No. 5,352,607 (1994)). Both are full-length proteins (134 kDa and 95 kDa, respectively) and both have been labeled with fluorescent dye or other labels for detecting chitin. Unfortunately, Chitinase VP1-derived chitin-detection reagent apparently stains cellulose in addition to chitin and therefore is non-specific (Linder et al., Applied and Environmental Microbiology 68:2503-2508 (2002)).

Detecting the presence of chitin is not a trivial task. For example, the chitinases described above may not be specific for chitin but may also recognize cellulose. This non-specific effect may be compounded by using non-specific dyes such as fluorescent calcofluor and lectins, both of which also bind other polysaccharides. In addition, chitinase degrades the substrate it is intended to detect. Consequently, a non-specific positive result may be obtained using these methods. There are also significant differences in chitin-binding specificity between different chitin-binding domains in the chitinases. Therefore, there is a need for improved reagents for detecting chitin specifically in diagnostic assays to detect the presence and amount of chitin in a sample and for research purposes that include studies on the location and amount of chitin in developing and mature organisms.

### SUMMARY

An embodiment of the invention describes a method for specifically detecting chitin and not cellulose in a sample. The method includes the steps of: (a) contacting the sample with a reagent comprising a chitin-binding domain (CBD) and optionally fused to a maltose-binding domain (MBD); and (b) detecting specifically whether chitin and not cellulose is present in the sample by the binding of CBD to chitin. Step (a) may be preceded by a bleaching step. The CBD may be selected from the class of CBM12 type CBDs. In a particular embodiment, the CBD is obtained from chitinase AI from Bacillus circulans.

In further embodiments, the CBD in the reagent is conjugated to a reporter. The detectable label may be any of a radioactive material, a fluorophore, a dye, an electron-dense compound, and an enzyme. The sample may include a plant tissue, an agricultural product, an animal tissue, a human tissue, a contact lens, a prosthetic device, or an air filter. The sample may further include an animal body fluid, a human body fluid, a plant fluid, potable water, or a beverage.

In a further embodiment, the contacting step (a) additionally comprises contacting the sample with a reagent such as an antibody to CBD or to a protein fused to CBD. Additionally, a detectable label may be included, for example, a radioactive material, a fluorophore, a dye, an electron-dense compound, and an enzyme.

In an embodiment of the invention, a kit is described that includes an immobilized CBD reagent and optionally instructions for use of the immobilized CBD reagent for detecting chitin. The kit may further include a soluble CBD carrier-protein (for example, maltose-binding protein (MBP) fusion molecule linked to a reporter. The reporter may include a rhodomaine or fluorescein dye. The CBD may be derived from chitinaseAI.

In an embodiment of the invention, a method for detecting chitin in a sample is provided that includes (a) obtaining an immobilized first CBD; (b) adding the sample and allowing any chitin in the sample to bind to the immobilized CBD; (c) adding a second CBD for binding the immobilized chitin of step (b) wherein the CBD is optionally linked to a protein carrier and a reporter molecule or to reporter molecule only and wherein the first CBD and the second CBD are obtained from the same or different chitinase; and (d) detecting the chitin in the sample. The second CBD may be linked to a carrier protein, wherein the carrier protein is maltose-binding protein. In addition, the chitin may be detected by means of a reporter selected from: a labeled antibody; a chromosphore such as a fluoroscein; rhodamine; or an enzyme such as alkaline phosphatase, peroxidase or beta galactosidase. Additionally, the first CBD may be immobilized by means of a chemical linker to a substrate, such as a bead, a gel, a filter, a column and a reaction vessel surface.

### LIST OF FIGURES

FIG. 1 shows specific chitin staining in C. elegans eggshell (Panel A) and pharynx (Panel B) using green fluorescent protein (GFP) labeled CBD. Arrows point to the stained chitin observed with fluorescent microscopy (top) or corresponding cell structure observed with DIC microscopy (bottom). Early stage embryos do not stain because they lack chitin. Likewise, late stage embryos do not stain because an additional protective layer has been created that obscures the chitin.

FIG. 2 shows specific staining for chitin obtained with fluorescein isothiocyanate (FITC) labeled MBP-CBD chitin probe.

FIG. 3 shows sequences of chitin-binding domains of Chitinase A1 (FIG. 3-1), Chitovibrin (FIG. 3-2) and Chitinase VP1 (FIG. 3-2).

### DETAILED DESCRIPTION OF SPECIFIC EMBODIMENTS

A highly specific and sensitive chitin-detecting method is described for detecting the presence of chitin in infections or contaminations by fungi or invertebrates in humans, animals, and plant resources. In particular, the method may be used for evaluating the presence and severity of fungal infection or invertebrates in humans, food and beverages. CBD can also be used as a research tool to detect chitin in organisms, such as parasites, fungi and protozoa.

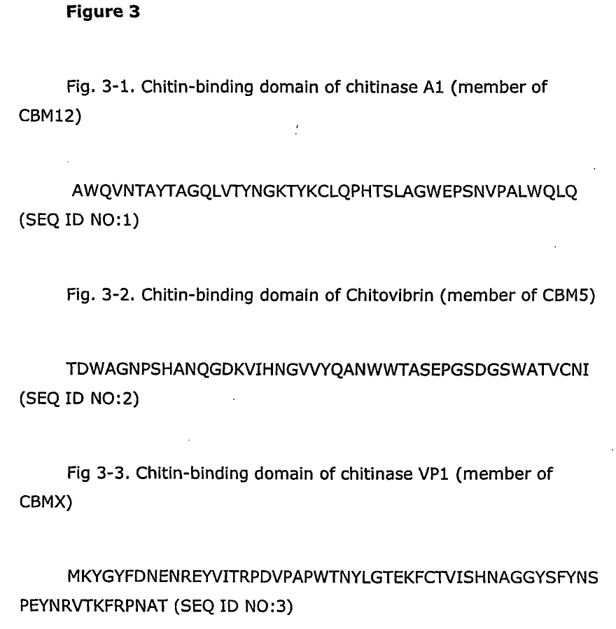

The term “Chitin Binding Domain” refers to a portion of a protein less than 70 amino acids, exemplified by the sequences in FIG. 3 and having a consensus sequence:

W-5-Y-12-5-H-7-P-S-L where the numbers in the consensus sequence refer to the numbers of amino acids separating the conserved residues.

In an embodiment of the invention, a method is provided that utilizes CBD, which has a high affinity for chitin to which it binds tightly and not to cellulose or other polysaccharides. CBD does not substantially degrade chitin. Moreover, the small size of CBD, its high affinity and specificity for chitin and the ability of a single chitin to stably bind multiple molecules of CBD means that a labeled CBD is a sensitive ligand for detecting chitin.

CBD can be readily produced in large amounts easily and rapidly from recombinant sources. It can also be obtained from native sources or by synthesis. The recombinant form may be a fusion or non-fusion protein, derivative or portion thereof. It is convenient but not required to form a fusion between CBD and a protein carrier such as MBP. In addition, the CBD may be fused to a protein reporter although chemical reporters may also be used.

CBD can be constructed and expressed as a fusion protein to which a reporter is attached to form the diagnostic reagent. For example, MBP has been made as an in-frame fusion with CBD and the conjugate purified as described in the NEB catalog (New England Biolabs, Inc., Beverly, Mass.) (Example 1). Although the method in Example 1 specified the CBD from chitinase AI, the method is applicable for cloning any CBD desired for which a sequence is known.

The reporter can be a protein such as MBP associated with CBD as a fusion protein or chemically coupled after synthesis. The chromophore can be coupled, for example, to a rhodamine or fluorescein. The reporter may alternatively be an enzyme such as alkaline phosphatase, peroxidase or betagalactosidase, which causes a color change in the presence of a suitable substrate according to the methods well known in the art.

CBD alone or a fusion protein containing a carrier protein and CBD can be used as an affinity tag for binding chitin-containing material. For example, the MBP-CBD can be cross-linked with a bifunctional reagent to a membrane, beads, filters or polymers known in the art. This will create a solid support that has high affinity for chitin. An example of this solid support is a CBD-coated magnetic bead (NEB catalog, New England Biolabs, Inc., Beverly, Mass.). The beads can be mixed with a chitin-containing solution and the magnetic particles to which the chitin has bound can then be pulled out of solution with a magnet. This simple enrichment step greatly aids in detection of chitin material in dilute solutions. The bound chitin can then be detected with a fluorescently labeled protein as previously described.

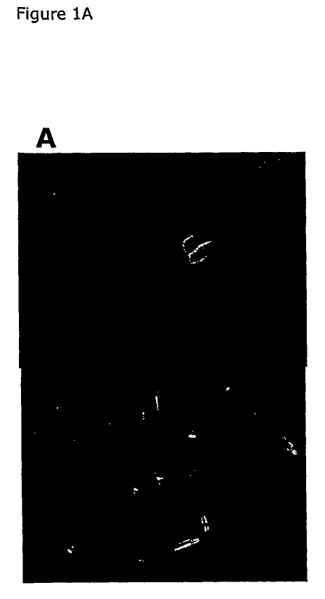

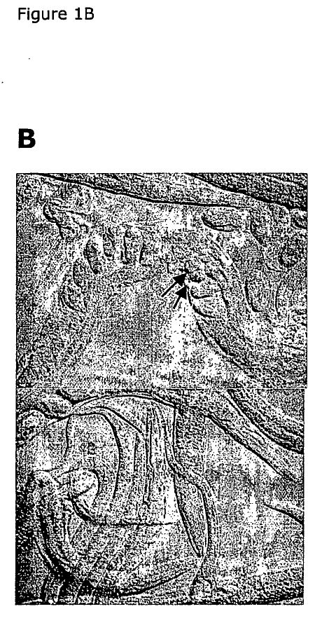

The labeled CBD diagnostic reagent can be used to detect and quantify chitin in whole organisms such as a free-living nematode, for example, Caenorabditis elegans and a fruit fly, for example, Drosophila melanogaster. (Examples 1-7, FIG. 1).

In a preferred embodiment, the chitin-detecting method utilizes the CBD from the C-terminal end of Chitinase Al derived from Bacillus circulans WL-12 (NEB catalog, New England Biolabs, Inc., Beverly, Mass.). This CBD was synthesized as a fusion protein consisting of E. coli MBP and CBDcA1 (MBP-CBDA1) in the Examples 2-7.

CBD from chitinase A1 has a high affinity for chitin and binds chitin in a similar manner but with greater specificity than the intact chitinase A1 from which it is derived (Hasimoto et al., J Bacteriol. 182: 3045-3054 (2000)). Recombinant CBD from chitinase A1 was found to bind chitin at a pH greater than pH3 but not to bind (GIcNAc)₆, ethylene glycol chitin, CM-chitin, soluble chitin, cellulose or starch. In contrast, intact chitinase A1 showed strong binding to chitin but also showed weak binding to cellulose and starch. Through mutagenesis, several key residues including Trp¹²² and Trp¹³⁴ were found to be important for binding (Watanabe et al., FEBS Lett. 494:74-78 (2001); Ferrandon et al., Biochem Biophys Acta. 1621:31-40 (2003)).

Analysis of the amino acid sequences of biochemically-characterized carbohydrate-binding modules (CBM) shows different types of CBDs. For example, chitinase A1 contains a CBD that belongs to the CBM12 group (Coutino, P. M. and Henrissat, B., in “Recent Advances in Carbohydrate Bioengineering”, eds. Gilbert, H. J. et al., pub. The Royal Society of Chemistry, Cambridge, U.K. pp. 3-12, 1999) while chitovibrin (U.S. Pat. Nos. 6,121,420 and 5,914,239) contains a putative CBD belonging to the CBM5 group of proteins. CBM12 and CBMS are distantly related in 3D structure and there is no significant sequence similarity. The chitinase VP1; (U.S. Pat. No. 5,352,607) contains a putative CBMX, which is less well characterized than either CBM12 or CBM 5 (FIG. 3). Moreover, neither CBM5 nor CBM12 showed any amino acid sequence homology to CBMX. In addition to a CBD, chitovibrin further includes an amino acid sequence that contains a region that is similar to Glyco-18 chitinase catalytic domain in chitinase A1, while the chitinase VP1 possesses a unrelated Glyco-hydro-20 chitinase catalytic domain. In all these proteins, the CBM is located in an amino acid region separated from the catalytic domain (FIG. 3).

In present embodiments of the invention, a CBD-based detection method was developed and its specificity and sensitivity illustrated by its ability to specifically detect chitin in the chitin-containing nematode C. elegans.

Chitin Assays

Chitin in a sample may be detected using direct assays or sandwich assays.

A direct assay involves measuring the binding of CBD linked to a reporter to chitin in a sample where the sample may be a liquid and then detecting the conjugate by means of the reporter after affinity immobilization or by size separation. Alternatively, the chitin may be detected by its immobilization to a substrate containing CBD.

In one embodiment, the sample is exposed to a solid substrate to which a CBD is attached, the CBD optionally being part of a fusion protein formed from the fusion of CBD with a protein carrier such as MBP, the CBD or CBD fusion protein being attached to the solid substrate via a chemical linkage group. The solid substrate may be magnetic beads, filter, column, various plastics or other material known in the art and described above to support a reaction. The amount of chitin is thus determined by a sandwich assay. In this way, chitin in a blood sample (after bleach treatment where applicable) present as a result of a pathogen (such as a nematode or fungus) can be detected in a cost-effective and convenient kit for rapid detection of chitin without expensive microscopy or other special instruments.

There are several approaches for linkage of protein to a solid substrate. The MBP-CBD protein can be linked to a bead or membrane with a variety of different reagents. One commercially available kit is the AminoLink Plus immobilization kit from Pierce Biotechnology Inc., Rockford, Ill. This approach links primary amines on proteins to a gel by reductive amination. This leads to a stable secondary amine linkage of the protein to the gel. A variety of other reagents can be used to link proteins to a support. They include gluteraldehyde, hydroxysuccinimide, tosyl chloride and cyanogen bromide. A discussion of these methods is given in Antibodies, A Laboratory Manual, by Ed Harlow David Lane, pub. Cold Spring Harbor Press, Cold Spring Harbor, N.Y., 1988, pp. 528 to 537.

The chitin-bound CBD can be visualized by existing techniques know in the art, such as directly labeling with fluorescent dyes, by forming a fusion of CBD with fluorescent proteins (such as GFP) or chemi-illuminant proteins (such as luciferase) or enzymes (such as peroxidase, alkaline phospatases, or beta-galactosidase), by the use of labeled antibodies or by radioactivity.

CBPs can be either examined using standard fluorescent or light microscopy (in situ detection), or measured quantitatively using appropriate enzyme coupled assays like in ELISA (quantitation). In situ detection will allow the visualization of the morphology of chitin-containing structures and also the whole organisms when in combination with other existing cell staining method, such as DNA-staining DAPI, which allows the nuclei to be visualized. On the other hand, quantitation methods described above will allow the estimation of relative abundance of chitin content (thus the abundance of chitin-containing organisms), for diagnosis of the severity of infection or contamination by fungi. It is envisaged that FACS sorting can be used to analyze a large number of different chitin-containing organisms stained with CBD to determine which species are present in a sample and their relative abundance. This approach would be useful in cases where possible infections by multiple fungal species are suspected.

In Examples 6-7, the detection of chitin in C. elegans is described using a recombinant fusion CBD-fusion protein where the fusion is between MBP and CBD. MBP provides a useful ligand for affinity purification of CBD where MBP reversibly binds amylose. The MBP is positioned with respect to CBD in such a way as not to interfere with CBD binding to chitin.

Using the staining reagent in Example 4, nematodes were incubated with crude bacterial lysates containing recombinant labeled CBD (GFP-CBD) for approximately 4 hours and then observed using microscopy. Specific staining was obtained in this short time frame and no significant increase was observed after longer staining. As shown in FIG. 1, strong staining was observed in the eggshells as predicted, and other structures such as the pharynx where chitin synthase expression has been reported. These examples confirm that CBD reagent allows the detection of chitin in a qualitative or quantitative manner by in situ staining of intact cells, tissues or whole organisms. Indeed, CBD reagent can be used to detect chitin in cell lysates and are the basis of chitin-containing products.

Diagnosis of Chitin-Containing Fungi

In embodiments of the invention, CBD was coupled to FITC, GFP or rhodamine (see Examples 1-3). The labeled CBD can then be used to stain a sample of an uncharacterized filamentous fungus or nematode that has been grown on nutrient agar plates and subsequently mounted on a solid substrate such as a microscope slide. Any accessible fungal proteins on the slide can be blocked with a 1% solution of bovine serum albumin (BSA) in PBS, and the slide washed with PBS (pH 7). Fifty μl of the CBD-FITC conjugate may be placed in contact with the specimen for thirty minutes, after which the slide is washed with PBS. PBS-glycerol is added to the slide, and the slide is photographed using standard fluorescence microscopy techniques, and filters specific for the fluorescence wavelength of fluorescein. The morphology of chitin stained samples will be visualized together with a counter stain such as DNA staining DAPI.

The CBD diagnostic system is effective in plant, animal, and human tissue sections with known fungal or nematode infections previously identified by standard tests. For example, tissue sections from animals with aspergillosis, cryptococcosis, and blastomycosis, tissue samples from human AIDS patients with candidosis, and tissue samples from maize or peanut plants, and their respective grains, infested with Aspergillus flavis, will be tested with a labeled CBD probe, or an anti-CBD antibody detected via a labeled secondary antibody, and examined under a light microscope or a fluorescent microscope. Similarly, tissue sections or blood can be tested from vertebrates or plants having filarial infections caused by parasitic nematodes.

In some cases, chitin may be partially or totally blocked by a polysaccharide capsule or other type of macromolecular coating found in some fungi such as that of Cryptococcus or in nematode eggs. However, chitin is extremely robust, hence, enzymatic digestions using proteases or polysaccharidases such as a glucanase or mannase can be used to permeate or remove the blocking layer before chitin detection. Alternatively, extreme treatment such as bleaching in a bleach solution (1% NaOCl, 0.5 M NaOH, or similar composition with higher or lower concentrations of individual chemical) (see Example 5) can be used to remove the masking layer.

Opportunistic fungal organisms are generally nonpathogenic in hosts with healthy immune systems. The validity of this diagnostic system will be confirmed with a variety of opportunistic fungal organisms, including those most commonly found in AIDS patients. Tissue preparation prior to staining will be conducted in accordance with standard procedures for fixed, paraffin-embedded tissues. The cells will be fixed with either methanol or 4% paraformaldehyde. The specimen will be embedded in paraffin and thin-sectioned. After a series of standard deparaffination procedures with xylene and a series of ethanol solutions at different concentrations, labeled CBD probe will be applied to pretreated specimens. Each specimen will be washed with PBS between additions of reagents. Several labeling probes will be investigated, to identify an optimal label for these tests. For example, the efficacy of CBD labeled with Peroxidase, FITC or rhodamine or other commercially available fluorescent dyes, anti-CBD IgG-Peroxidase or its fluorescent conjugate, peroxidase-antiperoxidase complex, and avidin-biotin will be evaluated. It may be advantageous to label the CBD directly with various labels known in the art, to eliminate the need for an anti-CBD antibody. For example, direct conjugates of fusion protein containing CBD with FITC or with horseradish peroxidase will be examined. Whether the chitin-specific binding protein or its antibody is labeled, detectable labels that may be used are labels those known in the art, including a radioactive material, a fluorophore, a dye, an electron-dense compound, or an enzyme. The variety of potential label possibilities broadens the potential applications of embodiments of this invention.

The diagnostic system can also be validated by testing chitinous materials in suspension, to demonstrate that the system also works in diagnosis with fluid samples of biological origin. A small filter unit (Spin-X, Vitaris, Baar, Germany), equipped with a polyvinylidene difluoride (PVDF, Immobilon, Millipore, USA) membrane pretreated with BSA will be used to retain chitinous materials, specifically swollen chitin in suspension. It should be feasible to retain chitinous fungal organisms, or their chitinous cell wall or yeast bud scar materials, on the membrane without interference by other proteins present in body fluids. Subsequently, the retained chitinous materials can be qualitatively detected by the diagnostic probe if desired.

A standard reconstruction assay may be used to detect pathogens or contaminants in a sample. This involves obtaining a preparation of target contaminant organisms (such as fungi or nematodes). The organisms are then mixed with normal human serum and increasing dilutions to determine the lowest detectable concentration of contaminating material.

Quantitative analysis can be achieved by detecting an appropriate labeled probe. To test that the specificity of the system, several control groups will be tested as well. These control groups will include serum contaminated with other known bacteria, viruses, or protozoa. Alternatively, direct labelling of CBD with visible dyes will be used for light microscopy and flow detection systems. The benefits of using CBD over chitinase or chitovibrin or other methods can be ascertained using an assay such as described above.

Nematode infections commonly involve skin, the intestines, stools and blood. For nematodes, the infectious worms may occur in small numbers in the blood following a circadian rhythm so that blood samples for diagnosis are commonly taken at night (night blood). The use of labeled CBD as described herein for detecting these small numbers of nematodes is significant. Blood samples may be treated with bleach and then analyzed in a sandwich assay using a CBD substrate and a CBD reagent described above. A similar procedure may be applied to the stools of a subject which can be treated with bleach and then tested in the above described sandwich assay to detect the eggs of Ascaris, for example.

In addition to the above described sandwich assay, the chitin can be detected by binding to a soluble labeled CBD fusion protein which can then be affinity purified and analyzed by microscopy or other means.

Similarly certain invasive fungal infections can be detected by analysis of the blood in the manner described above.

Correlation with other diagnostic procedures will be evaluated in a blind study with several control groups. Specimens from plants and animals with non-fungal infections, bacteremia, viral infection, and other disorders uncomplicated by fungal infections will also be tested as controls. Similar tests can be conducted with specimens from human patients, including AIDS patients and bone marrow transplant patients.

In an additional embodiment, a kit is provided that includes immobilized CBD and/or soluble labeled CBD-MBP and additionally includes instructions for determining the concentration of chitin in the sample.

All references cited herein are incorporated by reference.

### EXAMPLES

### Example 1

### Construction, Over-Expression and Purification of a Fusion Protein between E. coli Maltose-Binding Protein and CBDcA1 (MBP-CBDcA1)

DNA sequence encoding CBDcA1 from plasmid pTXB1 (NEB catalog #E6900S, New England Biolabs, Inc., Beverly, Mass.) was inserted inframe into the SacI site of pMAL-p2X (NEB, cat#E8000S, New England Biolabs, Inc., Beverly, Mass.) at the C-terminal of MBP. The resulting construct was transformed into E. coli ER2566 (New England Biolabs, Inc., Beverly, Mass.) and used to express a fusion protein of MBP-CBD. The fusion proteins were purified using an amylose column (NEB catalog #E8021S, New England Biolabs, Inc., Beverly, Mass.) according to manufacturer's instructions. The purified protein was concentrated to yield app. 13.7 mg/ml and determined to be substantially pure as it migrated as a single band in SDS-PAGE.

### Example 2

### Labeling MBP-CBDcA1 with Fluorescein

Approximately 65 μl of 13.7mg/ml (˜0.9 mg) purified MBP-ChBDchiA1 was reconstituted into 1 ml of PBS solution. 47.5 ul of FLUOS fluorescein at 2 mg/ml DMSO solution from Roche FITC labeling kit (catalog #1386093, Roche, Basel, Switzerland) was added and the labeling reaction was incubated for 2 hrs. The reaction mixture was passed through a pre-packed Sephadex G-25 column (5 cm long and 1.5 cm diameter of packed resin) supplied in the labeling kit to purify the free fluorescein from the labeled protein. The purified labeled protein was collected and yielded a protein concentration of about 0.6 mg/ml. The degree of labeling was calculated based on the absorption at OD²⁸⁰ (of protein) and OD⁴⁹⁵ (of fluorescein): Fluorescein to protein ratio (F/P)=(3.053×OD⁴⁹⁵)/(OD²⁸⁰−0.255×OD⁴⁹⁵), according to the instruction provided in the kit. A F/P around 2.9 was found for the above labeling, which indicated there were approximately 2.9 molecules of fluorecein on each fusion protein.

### Example 3

### Labeling MBP-CBDcA1 with Rhodamine

Approximately 1 mg at a concentration of 13.7 mg/ml, purified MBP-CBDcA1 was reconstituted with 927 μl of 0.1 M Na₂CO₃/NaHCO₃ buffer, pH 9.06, to yield a 1 ml solution. Rhodamine isothiocyanate (TRITC) (Sigma T-3163, Sigm-Aldrich, St. Louis, Mo.) 5 mg was dissolved in 2.5 ml DMSO and diluted to 1 mg/ml with 0.1 M Na₂CO₃/NaHCO₃ buffer, pH 9.06. 100 μl of 1 mg/ml TRITC or 2 mg/ml were added to each vial, which would achieve a labeling ration of 1:10 or 1:20 for IgG (150 kDa). The labeling reaction was incubated for 2 hrs. The reaction mixture was passed through Sephadex G-25 columns (5 cm long and 1.5 cm diameter of packed resin) to purify the non-reacted free TRITC from the labeled protein. The purified labeled protein had a concentration of 0.8 mg/ml. The degree of TRITC labeling was calculated based on the absorption at OD²⁸⁰ (of protein), OD⁵¹⁵ and OD⁵⁵⁵ (of rhodamine): F/P=(1.4×OD⁵⁵⁵)/(OD²⁸⁰×0.56−OD⁵¹⁵), according to the manufacture's instruction. F/P was around 2.5 for the 1:10 labeling reaction and 3.3 for the 1:20 labeling reaction, which indicated there were approximately 2.5 and 3.3 molecules of rhodamine on each fusion protein respectively.

### Example 4

### Construction of GFP-CBD Fusion Protein

Recombinant fusion proteins containing GFP as the fluorescent reporter were tested for CBD-based binding. (i) GFP was fused to the N-terminus of CBD (GFP-CBD), or (ii) GFP was contained within a nuclear hormone receptor (Nhr)-CBD fusion protein (Nhr-GFP-CBD). In addition, both fusion proteins contain a spacer sequence intein between GFP and CBD (NEB 2002/2003 Catalog, p. 164, Product No. 6900S, New England Biolabs, Inc., Beverly, Mass.). GFP-CBD and Nhr-GFP-CBD were each over-expressed in E. coli. Total protein lysates were prepared from bacterial cells expressing each fusion protein and directly used as the staining reagent.

### Example 5

### Preparation of C. elegans Sample for Staining

Wild type mixed-stage C. elegans were raised in standard nematode growth medium* (NGM) containing E. coli OP⁵⁰ (Brenner, Genetics 77:71-94 (1974)) and prepared for chitin staining as that reported for preparing C. elegans for antibody staining (Bettinger et al., Development 122:2517-2527 (1996)). After washing 3 times, the nematodes were adjusted to 0.9 ml of water and 1 ml of 2× Ruvkun fixation (2×: 160 mM KCl, 40 mM NaCl, 20 mM, ethylene glycol-bis (beta-aminoethyl ether) N,N,N′,N′-tetraacetic acid (EGTA), 10 mM spermidine HCl, 30 mM sodium piperazine-N,N′-bis[2-ethanesulfonic acid] (pH 7.4) (PIPES), 50% (v/v) methanol). After mixing, formaldehyde was added to a final concentration of 2%. The mixture was frozen in a dry ice/ethanol bath, thawed and incubated on ice for 3.5 hours with occasional inversion. The worms were washed with Tris/Triton buffer [100 mM Tris-Cl (pH 7.4), 1% (v/v) Triton X-100, 1 mM ethylenediaminetetraacetic acid (EDTA)], and incubated in Tris/Triton/1% β-mercaptoethanol at 37° C. with gentle agitation for 4 hours. The worms were washed with H₃BO₃ buffer [0.01 M H₃BO₃ (pH 9.2), 0.01 M NaOH], and incubated in H₃BO₃/10 mM dithiothreitol at 37° C. with gentle agitation for 15 minutes. The nematodes were washed with H₃BO₃ buffer, and incubated in H₃BO₃/0.3% (v/v) H₂O₂ at room temperature with gentle agitation for 15 minutes, and then washed in H₃BO₃ buffer and incubated in Buffer B [PBS, 0.1% (w/v) bovine serum albumin (BSA), 0.5% Triton X-100, 0.05% sodium azide, 1 mM EDTA] at room temperature for 30 minutes with gentle agitation. The nematodes were stored in Buffer A [Buffer A=Buffer B with 1% (w/v) BSA] at 4° C. for future staining with CBD-probes.

### Example 6

### Bleaching C. elegans Eggs Prior to Detection of Chitin in the Eggshell with the Chitin-Binding Domain

Gravid C. elegans on NGM plates were washed and subjected to a bleach treatment used to collect embryos (Brenner, Genetics, 1974 IBID). Briefly, 1 ml bleach solution (1% NaOCl, 0.5 M NaOH in H₂O) was mixed with a 200-300 μl pelleted nematodes and incubated for ˜10 min with occasional shaking and 10 ml of M9 buffer was added to dilute the bleach. The embryos were collect by centrifugation and washed 3 more times in M9 buffer*. Subsequently, the embryos were fixed in 2% formaldehyde solution in PBS and washed 3 times before staining.

* NGM media and M9 buffer are described in The nematode: Caenorhabditis elegans ed. William Wood, pub. Cold Spring Harbor Laboratory, Cold Spring Harbor, N.Y. 1988, pg 589.

### Example 7

### Staining of Whole C. elegans or Embryos with Fluorescent MBP-ChBDchiA1 or Bacterial Lysate Containing GFP-CBD and Detection of Chitin by Microscopy

Bleach treated nematodes (Example 5) or embryos (Example 6) were incubated with three dilutions of a stock solution of 0.6 mg/ml FITC- or Rhodamine-labeled MBP-ChBDchiA1 fusion protein as prepared in Examples 2 and 3 or with GFP-CBD fusion from Example 4 in Buffer A (1:10, 1:100 and 1:1000). At 1, 4 and overnight (˜18 hrs), the samples were mounted on to a 2% agar pad and inspected for staining pattern and signal intensity using a Zeiss Axiovert 200 M microscopy with corresponding barrier filters for detecting emission of light. (FITC: excitation filter BP 470/40 nm, emission filter BP 525/50 nm and Rhodamine: excitation filter BP 546/12 nm, emission filter BP 575-640 nm).

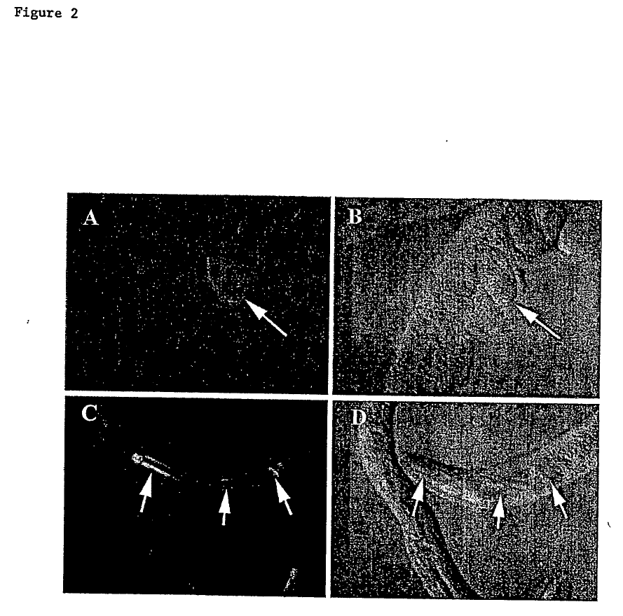

For FITC or Rhodamine conjugated CBD, staining after 1 hr gave minimal signal at 1:10 and 1:100 dilutions and no signal at 1:1000. At 4 hrs, all dilutions gave reliable staining (FIG. 2). At 1:10 dilution, high background signal was observed, due to the excess amount of stain and could be removed by washing. For GFP labeled CBD, staining was strong with 1:10 dilution and relatively weak signals at 1:100 dilution and negligible signal at 1:1000 dilutions (FIG. 1). Staining overnight did not significantly improve the fluorescent signals.

The staining was found in free embryos and some embryos inside the worm. Careful examination of stained embryos using high-resolution differential interference contrast (DIC) microscopy together with fluorescent microscopy revealed that the staining was in the eggshell surrounding the embryos (FIGS. 1 and 2). The staining was also found in the lining of the pharynx (FIGS. 1 and 2).

The embryos, prepared by bleaching, stained extremely brightly. Since chitin is insensitive to bleach, this approach selectively preserves chitin and remove any covering layer that mask the accessibility of the staining probe. This observation is useful for developing sensitive method for the detection of chitin in various organisms.

We claim:

1. A method for specifically detecting chitin and not cellulose in a sample, comprising the steps of:
(a) contacting the sample with a first reagent comprising a chitin-binding domain (CBD) and optionally fused to a maltose-binding domain (MBD); and
(b) detecting specifically whether chitin and not cellulose is present in the sample by the binding of CBD to chitin.

2. A method as recited in claim 1, wherein the CBD in the reagent is conjugated to a reporter.

3. A method as recited in claim 2, wherein the reporter is selected from the group consisting of a radioactive material, a fluorophore, a dye, an electron-dense compound, and an enzyme.

4. A method as recited in claim 1, wherein the sample comprises a plant tissue, an agricultural product, an animal tissue, a human tissue, a contact lens, a prosthetic device, or an air filter.

5. A method as recited in claim 1, wherein the sample comprises an animal body fluid, a human body fluid, a plant fluid, potable water, or a beverage.

6. A method as recited in claim 1, wherein the contacting step additionally comprises contacting the sample with a second reagent comprising an antibody to CBD or an antibody to a protein fused to CBD.

7. A method as recited in claim 6, wherein the first reagent additionally comprises a reporter.

8. A method as recited in claim 7, wherein the reporter is selected from the group consisting of a radioactive material, a fluorophore, a dye, an electron-dense compound, and an enzyme.

9. A method according to claim 1, wherein the CBD has a carbohydrate-binding module corresponding to CBM12.

10. A method according to claim 1, wherein step (a) is preceded by bleaching the sample.

11. A method according to claim 1, wherein the CBD is obtained from chitinase AI from Bacillus circulans.

12. A kit, comprising: an immobilized CBD reagent.

13. A kit according to claim 12, further comprising instructions for use of the immobilized CBD reagent for detecting chitin.

14. A kit according to claim 12, further comprising a soluble CBD carrier protein fusion molecule linked to a reporter.

15. A kit according to claim 14, wherein the carrier protein is MBP.

16. A kit according to claim 14, wherein the reporter is a rhodomaine or fluorescein dye.

17. A kit according to claim 13, wherein the CBD is derived from chitinase AI.

18. A method for detecting chitin in a sample, comprising:
(a) obtaining an immobilized first CBD;
(b) adding the sample and allowing any chitin in the sample to bind to the immobilized CBD;
(c) adding a second CBD for binding the immobilized chitin of step (b) wherein the CBD is optionally linked to a protein carrier and a reporter molecule or to reporter molecule only and wherein the first CBD and the second CBD are obtained from the same or different chitinase; and
(d) detecting the chitin in the sample.

19. A method according to claim 18, wherein the second CBD is linked to a carrier protein, wherein the carrier protein is MBP.

20. A method according to claim 19, wherein step (d) further comprises detecting the chitin by means of a labeled antibody.

21. A method according to claim 19, wherein the first CBD is immobilized by means of a chemical linker.

22. A method according to claim 19, wherein the first CBD is immobilized on a substrate selected from: a bead, a gel, a filter, a column and a reaction vessel surface.


SEQUENCE LISTING (3 sequences)

SEQ ID NO: 1 - protein, 45 aa, Bacillus circulans
AWQVNTAYTAGQLVTYNGKTYKCLQPHTSLAGWEPSNVPALWQLQ

SEQ ID NO: 2 - protein, 45 aa, Vibrio parahaemolyticus
TDWAGNPSHANQGDKVIHNGVVYQANWWTASEPGSDGSWATVCNI

SEQ ID NO: 3 - protein, 63 aa, Vibrio parahaemolyticus
MKYGYFDNENREYVITRPDVPAPWTNYLGTEKFCTVISHNAGGYSFYNSPEYNRVTKFRPNAT